(19) United States
(12) Patent Application Publication
(10) Pub. No.: US 2022/0313861 A1
(43) Pub. Date: Oct. 6, 2022

(54) A METHOD FOR REDUCING MALODOUR USING CUCURBITRILS

(71) Applicant: AQDOT LIMITED, Cambridge (GB)

(72) Inventors: Andrew HOWE, Cambridge (GB); Benjamin Thomas CHEESMAN, Cambridge (GB); Tim Chan SEEM, Cambridge (GB); Michael PEDDER, Cambridge (GB); Nicolas ESSELIN, Cambridge (GB); Roger COULSTON, Cambridge (GB); Johannes Gerardus DE ROOIJ, Ermelo (NL)

(73) Assignee: AQDOT LIMITED, Cambridge (GB)

(21) Appl. No.: 17/633,367

(22) PCT Filed: Aug. 5, 2020
(86) PCT No.: PCT/GB2020/051867

§ 371 Date: Feb. 7, 2022

(30) Foreign Application Priority Data
Aug. 5, 2019 (GB) 1911194.7

(51) Int. Cl. A61L 9/01; A61L 9/04

(52) U.S. Cl. CPC A61L 9/01; A61L 9/042; A61L 2101/44

(57) ABSTRACT

A method for reducing malodour includes the step of providing a solid composition including one or more cucurbiturils and derivatives and/or analogues thereof suspended in and/or bound by a thermoplastic and/or a thermosetting polymer medium, wherein the source of the malodour is external.

The invention relates to a method for reducing malodour, in particular to a method for reducing malodour comprising the step of providing a solid composition comprising one or more cucurbiturils and derivatives and/or analogues thereof suspended in and/or bound by a thermoplastic and/or a thermosetting polymer medium, wherein the source of the malodour is external.

WO 2017/141029 (Aqdot Limited) discloses use of a composition comprising a mixture of two or more cucurbiturils selected from cucurbit[5]urils, cucurbit[6]urils, cucurbit[7]urils, and cucurbit[8]urils, for counteracting malodour in a moist environment. The terms cucurbit[5]uril, cucurbit[6]uril, cucurbit[7]uril, and cucurbit[8]uril means a cucurbituril molecule formed from five, six, seven and eight glycoluril molecules respectively. The compositions can comprise additives selected from preservatives, dyes, pigments sequestrants and antioxidants, and may be provided in different forms including adsorbed on a substrate such as a fabric. The cucurbiturils may also be added to a product, such as consumer product for laundry, home or personal care, wherein the product is in the form of powders or granulates, tablets or single dose units, dispersions, emulsions, micro-emulsions, solutions, hydro-alcoholic products, wipes, sponges, aerosols or liquid dispensers, creams, balsam, polish, waxes and the like. The consumer product may, amongst other things, be an air freshener or an air filtration device.

WO 2018/037209 (Aqdot Limited) discloses a stable suspension composition comprising cucurbituril particles suspended in a medium. The medium can be a wax. The composition may further comprise a suspending agent selected from a multitude of polymers such as polyvinyl alcohol. The composition may further comprise additives selected from the group consisting of surfactants, biocides, viscosifying agents, antioxidants, chelating agents, wetting agents, deposition agents, foam control agents, fragrances, solvents, dies, pigments, antiperspirant, and conditioning agents. The composition may form part of a consumer product as described above including a candle, wherein the product is in the form of powders or granulates, tablets or single dose units, wipes, sponges, compressed gas, aerosols or liquid dispensers, creams, balsam, polish, waxes and the like. The composition of WO 2018/037209 may also be applied to an inanimate surface such as a kitchen or bathroom surface, or the surface of a granule or bead.

WO 2016/185209 (Aqdot Limited) discloses an epoxy composition comprising a cucurbituril complexed to a curative. Thus in one example, a composition comprising the curing agent 1,4-diaminobutane is at least partially complexed with cucurbit[8]uril, and bisphenol A diglycidyl ether showed a slower rate of curing on storage, i.e., was more stable, than a comparative composition without cucurbit[8]uril.

US 2018/0247632 (Henkel AG & Co., KGAA) discloses a hot melt composition suitable for damping applications, preferably sound deadening applications, and with low release of volatile organic compounds at the application temperature, the composition comprising a poly-alpha-olefin, an elastomeric styrene based copolymer, a tackifier, and a macrocycle. Macrocycles can be selected from cyclodextrin, calixarene and cucurbituril. In one example, it was demonstrated that a composition comprising C3/C2 poly-alpha-olefin, styrene-isoprene-styrene copolymer, alkyl phenolic resin (tackifier), graphite filler and beta-cyclodextrin released less volatile organic compounds from the product at 100 degrees centigrade in a “Fogging Test” than a comparative composition without beta-cyclodextrin.

There is thus still a need to provide a method for reducing malodour comprising the step of providing a solid composition, wherein the source of the malodour is external, in particular, wherein the solid composition comprises cucurbituril and/or a derivative and/or analogue thereof, and wherein the solid composition is in the form of an isolated film or a coating adhered to an inanimate surface. Surprisingly, it has been observed that cucurbituril retains its ability to reduce malodour wherein the source of the malodour is external despite being suspended in a thermoplastic and/or a thermosetting polymer medium. Furthermore the presence of the thermoplastic and/or a thermosetting polymer medium reduces or eliminates the technical problem of dusting of cucurbituril particulate powder.

### SUMMARY OF THE INVENTION

In a first aspect of the invention, a method for reducing malodour is provided, the method comprising the step of providing a solid composition comprising one or more cucurbiturils and derivatives and/or analogues thereof suspended in and/or bound by a thermoplastic and/or a thermosetting polymer medium, wherein the source of the malodour is external.

The term “malodour” means, for the purposes of this specification, an unpleasant or unwanted odour frequently encountered in everyday life and has a variety of origins. Typical malodours include odours that emanate from uncontrolled industrial activity, cleaning products including disinfectants, from human and pet body such as perspiration and excretion, from the kitchen (including but not limited to food and beverages) and from food processing, from tobacco smoke, and from mould. Some of the most disturbing malodours for the human being are sweat, faecal, urine, wet pet, cooking odours, especially garlic, cabbage, fish and onion, and the like. Malodours may also emanate from the fatty acid and fatty acid derivatives present in consumer products, for example in soaps, detergents, shampoos, and conditioners. Other examples of particularly undesirable malodours are those produced by depilatory creams (sulphur compounds). All of these malodours are particularly pungent.

The malodour to be reduced may be produced by a mixture of malodour-producing molecules. The malodour is reduced by a solid composition comprising one or more cucurbiturils and derivatives and/or analogues thereof.

Malodour reduction is achieved through complexation of the malodour-producing molecules with the one or more cucurbiturils and derivatives and/or analogues thereof.

The term “solid” means, for the purposes of this specification, solid at temperatures of up to at least 80, preferably at least 100, more preferably at least 120 degrees centigrade and for a thermoplastic polymer, having a glass transition temperature of at least 80, preferably at least 100, more preferably at least 120 degrees centigrade.

The term “bound by” means, in the context of this specification, that, in the context of the invention, the one or more cucurbiturils and derivatives and/or analogues thereof are bound together by, but not necessarily suspended in, the thermoplastic and/or a thermosetting polymer medium. Thus in one embodiment, the one or more cucurbiturils and derivatives and/or analogues thereof may protrude from the thermoplastic and/or a thermosetting polymer medium. In another embodiment, the one or more cucurbiturils and derivatives and/or analogues thereof, typically in the form of particles, is in the form of particles agglomerated together.

The term “external” means, for the purposes of this specification, external to the solid composition. Thus the solid composition is not itself a source of malodour.

In a second aspect of the invention, a solid composition is provided, the solid composition comprising one or more cucurbiturils and derivatives and/or analogues thereof suspended in and/or bound by a thermoplastic and/or a thermosetting polymer medium, the solid composition further comprising one or more fragrance molecules.

In a third aspect of the invention, a solid composition comprising one or more cucurbiturils and derivatives and/or analogues thereof bound by a thermoplastic and/or a thermosetting polymer medium is provided, wherein when the solid composition is in the form of a coating adhered to an inanimate surface or an agglomerate, the composition comprises more than 10, preferably more than 25, more preferably at least 50, more preferably at least 75% w/w one or more cucurbiturils and derivatives and/or analogues thereof and optionally one or more of carbon black and/or inorganic pigment and/or pigment extender and preferably no more than 95, more preferably no more than 98, most preferably no more than 99% w/w one or more cucurbiturils and derivatives and/or analogues thereof and optionally one or more of carbon black and/or inorganic pigment and/or pigment extender, wherein the solid composition comprises at least an effective amount of the one or more cucurbiturils and derivatives and/or analogues thereof.

### BRIEF DESCRIPTION OF THE FIGURES

The invention is described with reference to the Figures which show in:

FIG. 1 reduction in n-butyric acid concentration compared to control (cardboard or sponge substrate or n-butyric acid) (R) (%) versus mass of mixed (unsubstituted) cucurbituril (g) (PVOH=polyvinyl alcohol, LMW=low molecular weight, HMW=high molecular weight, CB=cucurbituril, paper=cardboard); and

FIG. 2 reduction in n-butyric acid concentration compared to control (cardboard or sponge substrate or n-butyric acid) (R) divided by mass of mixed (unsubstituted) cucurbituril (%/g) versus mass of mixed (unsubstituted) cucurbituril (g) (PVOH=polyvinyl alcohol, LMW=low molecular weight, HMW=high molecular weight, CB=cucurbituril, paper=cardboard).

### DETAILED DESCRIPTION OF THE INVENTION

In one aspect of the invention, a method for reducing malodour is provided, the method comprising the step of providing a solid composition comprising one or more cucurbiturils and derivatives and/or analogues thereof suspended in and/or bound by a thermoplastic and/or a thermosetting polymer medium, wherein the source of the malodour is external.

In order to prepare the solid compositions of the first second and third aspects of the invention, typically pellets or particulate powder of thermoplastic and/or one or more thermosetting polymer precursors are admixed with the one or more cucurbiturils and derivatives and/or analogues thereof in the form of particulate powder or agglomerated particles at room temperature (typically 20-25 degrees centigrade) at atmospheric pressure. In one embodiment, liquid solvents or carriers, such cyclohexane or water, may be added together with optional excipients such as pigments, fillers such as talc or diatomaceous earth, dispersing agents, adhesion promoters, and biocides, thereby to form a liquid coating or paint which is then applied to an inanimate surface. Thermosetting precursors include cross-linking agents added in order to crosslink other thermosetting polymer precursors thereby to form a network. Alternatively the thermoplastic and/or one or more thermosetting polymer precursors and one or more cucurbiturils and derivatives and/or analogues thereof are prepared separately in liquid solvents or carriers before subsequent combination to form a liquid coating or paint. In one embodiment, one or more cross-linking agents and the other thermosetting polymer precursors are prepared separately for subsequent combination to form a liquid coating or paint. The coating or paint must be liquid at application temperature and can be applied to an inanimate surface by any appropriate method including but not limited to spraying, brushing, rolling, dipping or roll-to-roll coating. The liquid solvents or carriers then evaporate, either at room or elevated temperature (above room temperature) at atmospheric pressure, thereby to produce the solid composition of the invention. Typically elevated temperature or radiation, such as ultraviolet light or electron beam, is required to cure the one or more thermosetting polymer precursors.

The term “liquid” means, for the purposes of this specification, liquid at temperatures greater than or equal to 5, and less than or equal to 400, 300, 250, 200, 150, more preferably less than or equal to 100 degrees centigrade.

In another embodiment, preparation of the solid compositions of the first second and third aspects of the invention does not involve liquid solvents or carriers. In this embodiment, the mixture of thermoplastic and/or one or more thermosetting polymer precursors admixed with the one or more cucurbiturils and derivatives and/or analogues thereof can be coated onto inanimate surface through electrostatic forces, for example, using powder coating or fluidized bed techniques, or may be passed through a die to provide the desired final shape, for example to form an isolated film. Elevated temperature (above room temperature) is required to cure the one or more thermosetting polymer precursors as well as fuse any surface coating whether based on a thermoplastic or thermosetting polymer medium or to process the mixture through a die. For thermoplastic polymers, the elevated temperature must be at least above the glass transition temperature, preferably above the melting temperature of the polymer. When the solid composition is in the form of an isolated film, the film can optionally be subsequently laminated onto an inanimate surface.

It has been observed that in order to form an isolated film, the mixture of thermoplastic polymer admixed with the one or more cucurbiturils and derivatives and/or analogues thereof preferably comprises less than 10, 7, 5, 2, 1% w/w water in order to reduce the number of holes that appear in the resulting isolated film due to evaporation of water.

The preparation of the solid compositions of the first second and third aspects of the invention in the form of agglomerates is described in Example 3.

Preferably the cucurbituril is selected from any one of the group consisting of cucurbit[5]uril, cucurbit[6]uril, cucurbit[7]uril, cucurbit[8]uril, and a mixture thereof. A derivative of a cucurbituril is a structure having one, two, three, four or more substituted glycoluril units. A substituted cucurbituril compound may be represented by the structure below:

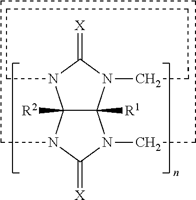

wherein n is an integer between 4 and 20; and for each glycoluril unit each X is O, S or NR³, and —R¹ and —R² are each independently selected from —H and the following optionally substituted groups R³, —OH, —OR³, —COOH, —COOR³, —NH₂, —NHR³ and —N(R³)₂, wherein —R³ is independently selected from C₁₋₂₀ alkyl, C₆₋₂₀carboaryl, and C₅₋₂₀ heteroaryl, or where —R¹ and/or —R² is —N(R³)₂, both —R³ together form a C₅₋₇ heterocyclic ring, or together —R¹ and —R² are C₄₋₆alkylene forming a C₆₋₈carbocyclic ring together with the uracil frame.

In one embodiment, one of the glycoluril units is a substituted glycoluril unit. Thus, —R¹ and —R² are each independently —H for n-1 of the glycoluril units. In one embodiment, n is 5, 6, 7, 8, 9, 10, 11 or 12. In one embodiment, n is 5, 6, 7 or 8. In one embodiment, each X is O. In one embodiment, each X is S. In one embodiment, R¹ and R² are each independently H.

In one embodiment, for each unit one of R¹ and R² is H and the other is independently selected from —H and the following optionally substituted groups —R³, —OH, —OR³, —COOH, —COOR³, —NH₂, —NHR³ and —N(R³)₂. In one embodiment, for one unit one of R¹ and R² is H and the other is independently selected from —H and the following optionally substituted groups —R3, —OH, —OR³, —COOH, —COOR³, —NH₂, —NHR³ and —N(R³)₂. In this embodiment, the remaining glycoluril units are such that R¹ and R² are each independently H.

Preferably —R³ is C₁₋₂₀alkyl, most preferably C₁₋₆alkyl. The C₁₋₂₀alkyl group may be linear and/or saturated. Each group —R³ may be independently unsubstituted or substituted. Preferred substituents are selected from: —R⁴, —OH, —OR⁴, —SH, —SR⁴, —COOH, —COOR⁴, —NH₂, —NHR⁴ and —N(R⁴)₂, wherein —R⁴ is selected from C₁₋₂₀alkyl, C₆₋₂₀carboaryl, and C₅₋₂₀heteroaryl. The substituents may be independently selected from —COOH and —COOR⁴.

In some embodiments, —R⁴ is not the same as —R³. In some embodiments, —R⁴ is preferably unsubstituted.

Where —R¹ and/or —R² is —OR³, —NHR³ or —N(R³)₂, then —R³ is preferably C₁₋₆alkyl. In some embodiments, —R³ is substituted with a substituent —OR⁴, —NHR⁴ or —N(R⁴)₂. Each —R⁴ is C₁₋₆alkyl and is itself preferably substituted.

A variant of cucurbituril may include a structure having one or more repeat units that are structurally analogous to glycoluril. The repeat unit may include an ethylurea unit. Where all the units are ethylurea units, the variant is a hemicucurbituril, for example hemicucurbit[12]uril:

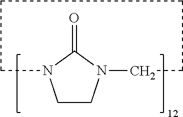

Preferably, the concentration of cucurbit[5]uril is from about 0 to about 99, more particularly from about 0.1 to about 75, more particularly from about 0.5 to about 50, more particularly from about 1 to about 30, more particularly about 1 to about 25, more particularly from about 1 to about 20% by weight, based on the total weight of cucurbituril in the composition

Preferably, the concentration of cucurbit[6]uril is from about 0.1 to about 99, more particularly from about 1 to about 75, more particularly from about 5 to about 60, more particularly from about 20 to about 55, more particularly from about 35 to about 55% by weight, based on the total weight of cucurbituril in the composition.

Preferably, the concentration of cucurbit[7]uril is from about 0.1 to 99, more particularly from about 5 to about 75, more particularly from about 10 to about 60, more particularly from about 20 to about 45% by weight, based on the total weight of cucurbituril in the composition.

Preferably, the concentration of cucurbit[7]uril is less than 45% by weight, based on the total weight of cucurbituril in the composition.

Preferably, the concentration of cucurbit[8]uril is from about 0.1 to 99, more particularly from about 0.5 to about 75, more particularly from about 1 to about 30, more particularly about 5 to about 25, more particularly from about 10 to about 20% by weight, based on the total weight of cucurbituril in the composition.

Preferably, the total concentration of cucurbit[5]uril, cucurbit[6]uril, cucurbit[7]uril, and cucurbit[8]uril in the composition is greater than 75, more particularly greater than about 90, more particularly greater than about 99% by weight, based on the total weight of cucurbituril in the composition.

Preferably, the composition comprises 1 to 17% by weight of cucurbit[5]uril, 30 to 50% by weight of cucurbit[6]uril; 20 to 37% by weight of cucurbit[7]uril, 10 to 27% by weight of cucurbit[8]uril, and less than 1% by weight of cucurbit[4]uril, cucurbit[9]uril and/or higher molecular weight cucurbiturils, based on the total weight of cucurbituril in the composition.

Typical malodour-producing molecules may be selected from nitrogen- and sulphur-containing molecules, preferably selected from the group consisting of allyl amine; methyl amine; ethyl amine; cyclobutyl amine (cyclobutanamine, urine), Cyclopentyl amine (cyclopentanamine); cyclohexyl amine (cyclohexanamine); cycloheptyl amine (cyclobutanamine); isopropylamine; butylamine; dibutylamine (N-butyl-1-butanamin); dimethyl ethanolamine (2-(dimethylamino)ethanol); methyl ethanolamine (2-(methylamino)ethanol); diethyl ethanolamine (2-(diethylamino)ethanol); diethylamine (N-methylethanamine, fishy); dipropyl amine (N-propyl-1-propanamine); diisopropylamine (N-isopropyl-2-propanamine); dimethyl acetamide (N,N-dimethylacetamide); ethyl methylamine (N-methylethanamine); ethyl propylamine (N-ethylpropanamide); trimethyl amine (fishy); triethylamine (fishy); ethylene diamine (1,2-ethanediamine, musty ammoniacal); propylene diamine (1,3-propanediamine); tetramethylenediamine (1,4-butanediamine, putrescine, foul); ethylene imine (aziridine, ammoniacal); morpholine (fishy); ethyl morpholine (4-ethylmorpholine, sour); pyrrolidine (semen); methyl ethyl pyridine (2-ethyl-3-methylpyridine); pyridine (burnt, sickening); vinyl pyridine (4-vinylpyridine, nauseating); skatole (3-methylindole, faecal); indole (faecal); cadaverine (pentane-1,5-diamine, putrid); hydrogen sulphide (rotten egg); allyl disulphide (3-(allyldisulfanyl)-1-propene, garlic); ethyl isothiocyanate (isothiocyanatoethane, pungent, mustard, garlic); allyl isothiocyanate (3-isothiocyanatoprop-1-ene, sulphurous); allyl mercaptan (2-propene-1-thiol, garlic, sulphurous); allyl sulphide (3-(allylsulfanyl)-1-propene; sulphurous); diallyl sulphide (3-(allylsulfanyl)-1-propene; sulphurous); dimethyl disulphide ((methylsulfanyl)ethane, unpleasant, garlic); dimethyl trisulphide (dimethyltrisulfane, foul); diethyl sulphide ((ethylsulfanyl)ethane, sulphurous); butyl sulphide (1-(butylsulfanyl)butane, garlic, violet); diethyl trisulfide (diethyltrisulfane, foul, garlic); ethyl methyl disulphide ((methylsulfanyl)ethane, sulphurous); phenyl sulphide (1,1′-sulfanediyldibenzene, sulphurous); ethyl mercaptan (1-ethanethiol, sulphurous); amyl mercaptan (1-pentanethiol); isoamyl mercaptan (3-methylbutane-1-thiol, sulphurous, onion); butyl mercaptan (1-butanethiol, skunk-like); isobutyl mercaptan (2-methylpropane-1-thiol, sulphurous, mustard); dodecyl mercaptan (1-dodecanethiol); carbon disulphide (methanedithione, disagreeable, sweet); dimethyl trithiocarbonate (dimethyl carbonotrithioate); and thiophenol mercaptan;

oxygen-containing five-member ring molecules, preferably selected from the group consisting of sotolone; and nor-sotolone;

saturated and unsaturated alkyl and hydroxyalkyl carboxylic acids, preferably selected from the group consisting of acetic acid, propionic acid, butyric acid, iso-valeric acid, n-valeric acid, 2-methyl-butyric acid, 3-methyl-2-hexenoic acid, and 3-methyl-3-hydroxy hexanoic acid; and cedryl acetate and naphthalenes.

Preferably, the thermoplastic medium is selected from the group consisting of linear low density polyethylene, low density polyethylene, medium density polyethylene, high density polyethylene, polyethylenes, polypropylenes, polyesters, polyethylene terephthalate, polyacrylate homo- and co-polymers, polymethacrylate homo- and co-polymers, poly(methyl methacrylate), poly(acrylonitrile butadiene styrene), polyamides, poly(lactic acid), poly(benzimidazole), polycarbonates, poly(ether sulfone), poly(oxymethylene), poly(etherether ketone), poly(etherimide), polystyrene, polyvinyl chloride, polyvinylidene fluoride, polytetrafluroethylene, celluloses, polysaccharides, polyvinyl alcohol, polyvinyl acetate, partially hydrolysed polyvinyl acetate, polyvinyl pyrrolidone, and mixtures thereof.

Preferably, the thermosetting medium is selected from the group consisting of polyurethanes, polyurea polyurethane hybrids, vulcanized rubber, polyacrylates, polymethacrylate, phenol-formaldehyde resins, urea-formaldehyde resins, melamine-formaldehyde resins, epoxy resins, benzoxazines and hybrids thereof with epoxy and phenolic resins, polyimides, polybismaleimides, cyanate ester resins, furan resins, silicone resins, vinyl ester resins, alkyd resins, and mixtures thereof.

Advantageously the composition additionally comprises one or more fragrance molecules.

In particular and in a second aspect of the invention, a solid composition is provide, the solid composition comprising one or more cucurbiturils and derivatives and/or analogues thereof suspended in a thermoplastic and/or a thermosetting polymer medium, the solid composition further comprising one or more fragrance molecules.

Common fragrance molecules include alcohols, aldehydes, ketones, lactones and O-heterocycles, ethers, acetals, ketals, N- and S— compounds, hydrocarbons and terpenes, and essential oils. Typical fragrance molecules are selected from (Z)-4-dodecenal (21944-98-9); 1-octen-3-ol (3391-86-4); 2,6-nonadienol (28069-72-9); 2-isobutyl-3-methoxypyrazine (24683-00-9); 2-nonenal (2463-53-8); 2-undecenal (2463-77-6); trans-4-decenal (65405-70-1); 8-decen-5-olide (32764-98-0); 9-decenol (13019-22-2); acetaldehyde, phenethyl propyl acetal (7493-57-4); 2,6,10-trimethylundec-9-enal (141-13-9); 10-undecenal (112-45-8); 2-methyl undecanal (110-41-8); allyl amyl glycolate (67634-00-8); allyl hexanoate (123-68-2); allyl phenoxyacetate (7493-74-5); alpha-amylcinnamaldehyde (122-40-7); alpha-damascone (43052-87-5); 3a,6,6,9a-tetramethyl-2,4,5,5a,7,8,9,9b-octahydro-1h-benzo[e][1]benzofuran (6790-58-5); 2-benzylideneheptanal (122-40-7); 1-(2-tert-butylcyclohexyl)oxybutan-2-ol (139504-68-0); amyl salicylate (2050-08-0); anisaldehyde diethyl acetal (2403-58-9); anisic aldehyde (123-11-5); benzaldehyde (100-52-5); benzyl acetate (140-11-4); beta-naphthyl methyl ether (93-04-9); ethyl 6-(acetyloxy)hexanoate (104986-28-9); beta-damascone (23726-92-3); beta-ionone (14901-07-6); 4-t-butylbenzenepropionaldehyde (18127-01-0); 8-methyl-1,5-benzodioxepin-3-one (28940-11-6 35783-05-2); 3-methyl-5-propylcyclohex-2-en-1-one (3720-16-9); cis-3-hexen-1-ol (928-96-1); cis-6-nonenal (2277-19-2); citral (5392-40-5); citronellal (106-23-0); citronellol (106-22-9); citronellyl oxyacetaldehyde (7492-67-3); dodecanenitrile (2437-25-4); coumarin (91-64-5); 2,6-nonadien-1-ol (7786-44-9); damascenone (23726-93-4); 2-pentyl cyclopentanone (4819-67-4); delta-damascone (57378-68-4); dihydro myrcenol (18479-58-8); dimethylbenzyl carbinyl acetate (151-05-3); diphenyl ether (101-84-8); 4-(octahydro-4,7-methano-5h-inden-5-ylidene)butanal (30168-23-1); 1-(5,5-dimethyl-1-cyclohexenyl)pent-4-en-1-one (56973-85-4); (z)-3-methyl-5-(2,2,3-trimethyl-1-cyclopent-3-enyl)pent-4-en-2-ol (67801-20-1); ethyl 2-methylbutyrate (7452-79-1); ethyl 2-methylpentanoate(39255-32-8); ethyl butyrate (105-54-4); ethyl n-ethylanthranilate (38446-21-8); ethyl trans-2,cis-4-decadienoate (3025-30-7); ethyl vanillin (121-32-4); ethyl vinyl ketone (1629-58-9); eucalyptol (470-82-6); eugenol (97-53-0); methyl 2,4-dihydroxy-3,6-dimethylbenzoate (4707-47-5); farnesene (alpha and beta) (502-61-4); fixolide (1506-02-1); tricyclodecenyl propionate (68912-13-0); 3-(3-propan-2-ylphenyl)butanal (125109-85-5); 2-butan-2-ylcyclohexan-1-one (14765-30-1); ethyl 2-(2-methyl-1,3-dioxolan-2-yl)acetate (6413-10-1); gamma-decalactone (706-14-9); gamma-undecalactone (104-67-6); geranyl acetate (105-87-3); 3,7-dimethyloct-6-enenitrile (5146-66-7); hexyl salicylate (6259-76-3); isoamyl acetate (123-92-2); isobutyl angelate (7779-81-9); isobutyl-quinoline (93-19-6); isoeugenol (97-54-1); isomethyl-alpha-ionone (127-51-5); isopropyl quinoline (137-79-5); tricyclodecenyl acetate (5413-60-5); 1-methyl-2-1,2,2-trimethyl-3-bicyclo[3.1.0]hexanyl]methyl]cyclopropyl]methanol (198404-98-7); 1-carvone (6485-40-1); (z)-3-hexen-1-yl methyl carbonate (67633-96-9); 3-(4-tert-butylphenyl)butanal (80-54-6); limonene (138-86-3, 7705-14-8); linalool (78-70-6); 3-methyl-7-propan-2-ylbicyclo[2.2.2]oct-2-ene-5-carbaldehyde (67845-30-1); 2,6-dimethylhept-5-enal (106-72-9); trans-2-dodecenal (20407-84-5); methyl cinnamate (103-26-4); (4-propan-2-ylcyclohexyl)methanol (5502-75-0); methyl 2-heptyne carbonate (111-12-6); methyl hexyl ketone (111-13-7); methyl octyne carbonate (111-80-8); 6,6-dimethoxy-2,5,5-trimethylhex-2-ene (67674-46-8); methyl salicylate (119-36-8); nerol oxide (1786-08-9); octanal (124-13-0); 1-naphthalen-1-ylethanone (941-98-0,93-08-3); (2r,4s)-2-methyl-4-propyl-1,3-oxathiane (59323-76-1); 2-cyclohexylidene-2-phenylacetonitrile (10461-98-0); 2-methyl-4-methylidene-6-phenyloxane (30310-41-9); 2-cyclohexyl-1,6-heptadien-3-one (313973-37-4); phenyl ethyl alcohol (60-12-8); 2-phenoxy ethanol (122-99-6); 3-(7,7-dimethyl-4-bicyclo[3.1.1]hept-3-enyl)propanal (33885-51-7); (e)-3,3-dimethyl-5-(2,2,3-trimethyl-3-cyclopenten-1-yl)-4-penten-2-ol (107898-54-4); gamma nonalactone (104-61-0); p-tolyl phenylacetate (101-94-0); (e)-2-ethyl-4-(2,2,3-trimethyl-1-cyclopent-3-enyl)but-2-en-1-ol (28219-61-6); 4-(p-hydroxyphenyl)-2-butanone (5471-51-2); 4-methyl-2-(2-methylprop-1-enyl)oxane (16409-43-1); m-(isocamphyl-5)cyclohexanol (66068-84-6); trans-2,cis-6-nonadienal (557-48-2); trans-2-hexenal (6728-26-3); trans-2-hexenyl 2-methylbutyrate (94089-01-7); trans-anethole (4180-23-8); 2,4-dimethylcyclohex-3-ene-1-carbaldehyde (68039-49-6); trimofix o (144020-22-4 68610-78-6); undeca-1,3,5-triene (16356-11-9); 4-methyldec-3-en-5-ol (81782-77-6); vanillin (121-33-5); and decahydrospiro(furan-2(3h),5′-(4,7)methano(5h)indene) (68480-11-5); and nona-2,6-dienenitrile (67019-89-0).

Typically, the solid composition may comprise 0.01 to 15, preferably 0.01 to 10, most preferably 0.01 to 5% w/w one or more fragrance molecules.

The solid composition of the first aspect of the invention comprising a fragrance molecule and the solid composition of the second aspect of the invention remain substantially odourless because the fragrance molecule is complexed with cucurbituril. However, decomplexation and hence release of the fragrance molecule may be achieved by the action of a malodour-producing molecule complexing with cucurbituril and hence displacing the fragrance molecule. Thus the solid composition not only reduces malodour but also releases a fragrance. One advantage of the solid composition is that molecular exchange of the fragrance molecule with the malodour molecule can take place even in humid conditions (at least 40% relative humidity at room temperature). Decomplexation and release of the fragrance molecule may also be accomplished by exposure to moisture or liquid water, evaporation, heat and molecular exchange.

In one embodiment, the trigger for decomplexation and release of the fragrance molecule is water activity which is increased by increasing ambient relative humidity. The water activity may increase in such extent that water molecules will tend to bind to the cucurbiturils and displace part the fragrance molecules into the air. Contacting the solid composition with water is another way to increase the water activity.

In another embodiment, the trigger for decomplexation and release of the fragrance molecule is evaporation or heat. Evaporation and heat are related to each other via the well-known temperature dependence of the vapour pressure. When the interplay of evaporation and heat is taken as the driving force for fragrance molecule release, selection of the fragrance molecules may be achieved by considering the vapour pressure of each fragrance molecule. For example, for slow release at room temperature, fragrance molecules having vapour pressure higher than 0.1 mm Hg at 20 degrees centigrade may be selected, while under heat-induced release conditions, for example at 100 degrees centigrade or more, fragrance molecules having lower vapour pressure may offer better results. The person skilled in the art will appreciate the diversity of fragrance molecules in terms of vapour pressures and odour characteristics that are left open to creation, when considering evaporation and heat as triggers.

In another embodiment, the trigger for decomplexation and release of the fragrance molecule is molecular exchange-mediated release of fragrance molecules. It has been observed that complexes of fragrance molecules and cucurbituril, and especially complexes where the fragrance molecule comprises oxygen heteroatoms, are generally weak compared to complexes of cucurbituril and nitrogen-containing or sulphur-containing molecules, or more particularly complexes of cucurbituril and cationic molecules. Thus the compound for triggering decomplexation and release of the fragrance molecule may be selected from metal ions and neutral, cationic, zwitterionic, amphoteric and/or cationic nitrogen-containing, sulphur-containing and/or oxygen-containing substances. Contact between the solid composition and the trigger compound may be achieved by a variety of means, for example, the solid composition of the invention and the trigger compound may be supplied as a water-dispersible solid form, such as a powder or granulate, which when dispersed in water releases the trigger thereby decomplexation and release of the fragrance molecule. Alternatively, in-situ formation of the trigger compound may occur following a change of pH.

Typical trigger compounds include, but are not limited to sulfonium derivatives and S-heterocyclic materials, amines and polyamines, and their quaternized forms; imines and polyimines, such as polyethyleneimines and other polyalkylene-imines, and their quaternized forms; amino-silicones, such as aminoalkyl-dimethicone; hydroxy amines; cationic surfactants, such as alkylammonium surfactants having one or two alkyl chain comprising from about 16 to about 22 carbon atoms and two to three alkyl moieties having chain length from 1 to about 4 carbon atoms, optionally having one or more hydroxyl group, or hydroxyalkyl moieties having about 1 to about 10 ethylene oxide moieties; N-heterocyclic materials, such as oxazoline derivatives, piperazine derivatives, pyridine, bipyridin and polypyridin derivatives, amino-pyridinium derivatives, cyclam derivatives, pyrrole derivatives, imidazole derivatives, and the like, and mixture thereof; fused polycyclic materials comprising said N-heterocyclic materials; and mixture thereof.

The solid composition of the first and second aspects of the invention may be in the form of an isolated film, a coating adhered to an inanimate surface, porous substrate or an agglomerate. The inanimate surface may be in the form of a porous substrate. Alternatively, the porous substrate itself may be produced by aerating the mixture of thermoplastic and/or one or more thermosetting polymer precursors admixed with the one or more cucurbiturils and derivatives and/or analogues thereof with a suitable gas either generated in-situ or ex-situ such as air, nitrogen or carbon dioxide when in the liquid state and allowing to cool whilst aerated. The isolated film can optionally be subsequently laminated onto an inanimate surface. The isolated film may be in the form of or form part of a refuse bin liner. The inanimate surface may be formed of any material suitable for supporting the solid composition, for example the inanimate surface may be, but not limited to, paper, wood, a plastics material, stone, ceramic, metal, a textile, and plaster. More specifically the inanimate surface or porous substrate may form part of a home or personal care product such as a feminine hygiene product, a diaper, an incontinency pad, a sanitary napkin, a shoe sole, or an air filter. The term “porous”, for the purposes of this specification, means that that liquid or gas may pass through, for example the substrate, via interstices within that substrate.

Preferably when the solid composition of the first and second aspects of the invention is in the form of an isolated film, the composition comprises 0.01 to 10, preferably 0.1 to 7.5, more preferably 0.5 to 5, more preferably 0.7 to 3% w/w one or more cucurbiturils and derivatives and/or analogues thereof.

Preferably when the solid composition of the first aspect of the invention is in the form of a coating, the composition comprises more than 10, preferably more than 25, more preferably at least 50, more preferably at least 75% w/w one or more cucurbiturils and derivatives and/or analogues thereof and optionally one or more of carbon black and/or inorganic pigment and/or pigment extender and preferably no more than 95, more preferably no more than 98, most preferably no more than 99% w/w one or more cucurbiturils and derivatives and/or analogues thereof and optionally one or more of carbon black and/or inorganic pigment and/or pigment extender, wherein the solid composition comprises at least an effective amount of cucurbiturils and derivatives and/or analogues thereof. The term “effective”, for the purposes of this specification, means, in the context of the amount of cucurbituril, that amount which is effective at reducing malodour.

Inorganic pigments and pigment extenders are in particulate form and well-known to the skilled person. Examples of inorganic pigments are iron oxide and titanium dioxide, and examples of pigment extenders are talc, diatomaceous earth, calcium carbonate and calcium sulphate.

When the solid composition of the first and second aspects of the invention is in the form of an isolated film, the one or more cucurbiturils and/or derivatives and/or analogues thereof are preferably in the form of particles or agglomerates of particles of D90 (using a microscope) D90 no larger than the film thickness. It has been observed that such particles or agglomerates of particles with D90 larger than the film thickness bridge the two opposing surfaces of the film thereby structurally weakening the film.

In a third aspect of the invention, a solid composition comprising one or more cucurbiturils and derivatives and/or analogues thereof bound by a thermoplastic and/or a thermosetting polymer medium is provided, wherein when the solid composition is in the form of a coating adhered to an inanimate surface or an agglomerate, the composition comprises more than 10, preferably more than 25, more preferably at least 50, more preferably at least 75% w/w one or more cucurbiturils and derivatives and/or analogues thereof and optionally one or more of carbon black and/or inorganic pigment and/or pigment extender and preferably no more than 95, more preferably no more than 98, most preferably no more than 99% w/w one or more cucurbiturils and derivatives and/or analogues thereof and optionally one or more of carbon black and/or inorganic pigment and/or pigment extender, wherein the solid composition comprises at least an effective amount of the one or more cucurbiturils and derivatives and/or analogues thereof.

In the Examples described below, reference to mixed (unsubstituted) cucurbiturils is reference to a mixture comprising 1 to 17% by weight of cucurbit[5]uril, 30 to 50% by weight of cucurbit[6]uril; 20 to 37% by weight of cucurbit[7]uril, 10 to 27% by weight of cucurbit[8]uril, and less than 1% by weight of cucurbit[4]uril, cucurbit[9]uril and/or higher molecular weight cucurbiturils, based on the total weight of cucurbituril, prepared in accordance with the process described in any one of Examples 5 to 7 of WO 2018/115822 (Aqdot Limited).

### Example 1: Film Comprising Cucurbituril

(a) Sample preparation

Pellets of linear low density polyethylene (LLDPE), obtained from SABIC (grade 318B) and milled to powder form, comprising 20% w/w mixed (unsubstituted) cucurbiturils (“cucurbituril masterbatch”) were prepared by high speed mixing of the cucurbiturils and LLDPE powder followed by passage through a twin-screw extruder at 150° C. The strand generated was passed through a die and then through rollers to air-cool before being cut into pellets. The pellets were placed in a convection oven at 100° C. to minimise moisture uptake.

LLDPE film was produced on a three metre vertical film tower by filling the hopper of the film tower with appropriate ratios of the “cucurbituril masterbatch” pellets and LLDPE pellets and extruding the mixture at 170° C. before passing the mixture through a circular die after which air was introduced to the mixture to blow a film. Films containing up to 10% w/w mixed (unsubstituted) cucurbiturils (50% w/w “cucurbituril masterbatch” and 50% w/w LLDPE) were produced with an approximate 35 micron film thickness.

(b) (Mal)odour reduction: Headspace-Gas Chromatography (GC-HS)

200 mg samples of the film were placed inside 20 mL headspace vials with an odour compound contained in a separate 1.5 mL vial. The odour compound was provided in the form of either 15 μL (microliter) of 1.5% w/v aqueous solution of trimethylamine or 20 μL (microliter) of 1% w/v aqueous solution of butyric acid.

Malodour concentration in the headspace of the headspace vials was determined using headspace-gas chromatography (GC-HS). The concentration of trimethylamine was determined using a 60 metre CP-Volamine column (Agilent Technologies) with samples equilibrated for 30 minutes in a headspace oven at 50° C. The concentration of butyric acid was determined using a 60 metre DB-wax column (Agilent Technologies) with samples equilibrated for 30 minutes in the headspace oven at 90° C. All measurements were performed in triplicate.

The concentration was determined by integrating the chromatography peak area detected at the characteristic retention time for each odour compound. Odour reduction was calculated as a measure of the effectiveness of the films at reducing the odour of each odour compound and was defined as the ratio of the area of the peak for each odour compound in the presence of the film comprising mixed (unsubstituted) cucurbiturils relative to that recorded in the presence of a control LLDPE film not comprising mixed (unsubstituted) cucurbiturils.

The results are summarised in Table 1 and show that the film comprising mixed (unsubstituted) cucurbiturils effectively reduces the odour of both trimethylamine and butyric acid as the concentration of mixed (unsubstituted) cucurbiturils increases between 0 and 10% w/w.

TABLE 1
(Mal)odour reduction performance of LLDPE blown films comprising
mixed (unsubstituted) cucurbiturils measured by GC-HS.

| Mixed cucurbiturils (% | Butyric acid | Standard deviation (%) | Trimethylamine | Standard deviation (%) for |
|---|---|---|---|---|
| w/w) | (% reduction) | for butyric acid | (% reduction) | trimethylamine |
| 0 | 0 | 12.4 | 0 | 6.0 |
| 1 | 16 | 4.2 | 24 | 5.8 |
| 2 | 57 | 6.6 | 48 | 4.4 |
| 3 | 66 | 4.3 | 70 | 4.2 |
| 5 | 74 | 3.3 | 83 | 2.8 |
| 10 | 89 | 3.2 | 94 | 7.5 |

(c) (Mal)Odour Reduction: Sensory Performance

Two 5×5 cm trimethylamine-loaded polycotton (a combination weave of cotton and polyester) swatches were prepared by adding 22.2 μL (microliter) of 45% w/v trimethylamine in water to each swatch. One swatch was placed into a 20 cm length tube of LLDPE (control) film and the other was placed into a 20 cm length tube of LLDPE film comprising either 2 or 10% w/w mixed (unsubstituted) cucurbiturils.

The ends of all the tubes of film were sealed with cable ties and each sealed tube placed into individual 10 L Nalophan sample bags which were subsequently sealed and inflated with compressed air and left to equilibrate at 20° C. and a relative humidity of 40-60% for one hour. The headspace of the Nalophan bags was then smelt in a blind pairwise comparison test by a trained six-person panel for (mal)odour intensity and hedonic tone.

Odour intensity, which is measured in conjunction with odour concentration, is the perceived strength of odour above its detection threshold. The odour was described on a seven point scale from not perceptible to extremely strong (6 extremely strong; 5 very strong; 4 strong; 3 distinct; 2 weak; 1 very weak; 0 not detectable). Odours can have different perceived intensity at the same concentration.

Hedonic tone measures the pleasantness of an odour which may change from pleasant to unpleasant with an increasing concentration, intensity and frequency. The analysis determines the concentration at which an odour becomes a nuisance and rated the odour on a nine point pleasant/unpleasant scale (+4 extremely pleasant; +3 very pleasant; +2 pleasant; +1 weakly pleasant; 0 neutral; −1 weakly unpleasant; −2 unpleasant; −3 very unpleasant; −4 extremely unpleasant).

The trained six-person panel was trained every month using a set of sniffing sticks with different levels of butanol and assessing the odour strength broadly in accordance with European Standard EN13725: Air quality—Determination of odour concentration by dynamic olfactometry. Each comparative experiment was conducted in triplicate.

The results are summarised in Tables 2 and 3 and show that for LLDPE film comprising 2% w/w mixed (unsubstituted) cucurbiturils, the difference between the two films for Repeat 1 was of low significance (0.01<P<0.05), for Repeat 2 was highly significant (P<0.005) and for Repeat 3 was significant (0.005<P<0.01). P is the probability that the results occurred randomly as calculated by a two-tailed t-test, whereas for LLDPE film comprising 10% w/w mixed (unsubstituted) cucurbiturils, the difference between the two films for all three Repeats was highly significant (P<0.005).

TABLE 2
Sensory (mal)odour intensity and hedonic tone comparison of LLDPE film comprising 2%
w/w mixed (unsubstituted) cucurbiturils versus LLDPE control film using trimethylamine as the
odour compound (SEM = standard error of the mean).

| | Mixed | | | | |
|---|---|---|---|---|---|
| | cucurbiturils | | | | SEM (hedonic |
| Experiment | (% w/w) | Intensity | SEM (intensity) | Hedonic Tone | tone) |
| Repeat 1 | 0 | 3.42 | 0.49 | -1.60 | 0.22 |
| | 2 | 1.17 | 0.48 | -0.40 | 0.22 |
| Repeat 2 | 0 | 3.40 | 0.47 | -2.00 | 0.41 |
| | 2 | 1.50 | 0.29 | -0.60 | 0.37 |
| Repeat 3 | 0 | 2.70 | 0.34 | -1.80 | 0.34 |
| | 2 | 0.90 | 0.37 | -0.70 | 0.40 |

TABLE 3
Sensory (mal)odour intensity and hedonic tone comparison of LLDPE film comprising 10%
w/w mixed (unsubstituted) cucurbiturils versus LLDPE control film using trimethylamine as the
odour compound (SEM = standard error of the mean).

| | Mixed | | | | |
|---|---|---|---|---|---|
| | cucurbiturils | | | | SEM (hedonic |
| Experiment | (% w/w) | Intensity | SEM (intensity) | Hedonic Tone | tone) |
| Repeat 1 | 0 | 4.00 | 0.45 | -2.75 | 0.36 |
| | 10 | 1.67 | 0.57 | -0.83 | 0.31 |
| Repeat 2 | 0 | 4.70 | 0.20 | -2.80 | 0.37 |
| | 10 | 1.60 | 0.40 | -0.70 | 0.30 |
| Repeat 3 | 0 | 3.58 | 0.20 | -2.33 | 0.21 |
| | 10 | 1.25 | 0.31 | -0.42 | 0.27 |

### Example 2: Coating of Mixed (Unsubstituted) Cucurbiturils and Polyvinyl Alcohol on a Planar or Porous Support

Cucurbituril has been shown to be an effective material for counteracting malodour and can be used in liquid form (suspension) enabling delivery as an aerosol. Cucurbituril can be effective in solid form but as a simple powder may suffer from drawbacks related to dusting, inhalation or unwanted deposition. The foregoing drawbacks are solved by immobilising cucurbituril on a substrate. Immobilisation may be achieved by combining cucurbituril with a binder and applying the resulting mixture onto a substrate as a coating. Other materials may be included within the coating, either to provide enhancements to the coated layer or to assist application of the coating.

(a) Sample Preparation

Water-based suspensions were prepared containing mixed (unsubstituted) cucurbituril and polyvinyl alcohol by combining equal masses of 50% w/w aqueous slurry of mixed (unsubstituted) cucurbituril with a 2.5% w/w aqueous solution of polyvinyl alcohol. The resultant composition comprised 1.25% w/w polyvinyl alcohol and 25% w/w mixed (unsubstituted) cucurbituril in water.

The 50% w/w aqueous slurry of mixed (unsubstituted) cucurbituril was prepared by adding water to mixed (unsubstituted) cucurbituril in the form of a powder and stirring the resulting mixture with a glass rod. Polyvinyl alcohol solutions in water were prepared by adding granules of polyvinyl alcohol to water under stirring and then heating to the mixture to 90 degrees centigrade until dissolution was complete. Two polyvinyl alcohol samples were used, both supplied by Sigma Aldrich and each with degree of hydrolysis 88% and with nominal molecular weights of 67 kDa (Mowiol 8−88) and 205 kDa (Mowiol 40−88) and are correspondingly referred to as low MW and high MW respectively. The viscosity at 1 s⁻¹ and 20 degrees centigrade of the 2.5% w/w polyvinyl alcohol solutions was 3.2 and 9.8 mPa·s for the low MW and high MW samples respectively.

1 cm by 5 cm cardboard swatches were manually coated with the mixed (unsubstituted) cucurbiturils and polyvinyl alcohol water-based suspensions by dipping, and were then then dried in a 45 degree centigrade oven overnight. 1 cm by 1 cm by 2 cm pieces of artificial sponge were loaded by immersion in the mixed (unsubstituted) cucurbiturils and polyvinyl alcohol water-based suspensions, the excess squeezed out, and then dried in a 45 degrees centigrade oven overnight.

The amount of mixed (unsubstituted) cucurbiturils and polyvinyl alcohol water-based suspensions and hence of mixed (unsubstituted) cucurbiturils on each substrate sample was determined by mass.

(b) (Malodour reduction: Gas Chromatography-Headspace Analysis

4 μL (microliter) of n-butyric acid (a model malodour compound) was added into a 20 mL headspace vial alongside each support. The malodour concentration was measured by Gas Chromatography-Headspace analysis (GC-HS). Analysis used a 60 metre DB-wax column (Agilent Technologies) with samples equilibrated for 30 minutes in the headspace oven at 90 degrees centigrade. 10 mL of headspace was extracted for analysis. All measurements were performed in triplicate.

The malodour concentration was determined by integrating the peak area detected at the characteristic retention time for n-butyric acid. The malodour reduction was calculated as the ratio of the malodour peak in the presence of the mixed (unsubstituted) cucurbituril-containing substrate, relative to the malodour peak recorded in the presence of the control sample (substrate with no mixed (unsubstituted) cucurbiturils).

i) Cardboard Substrate

The results are presented in Table 4 and expressed in terms of the percentage reduction (R) in n-butyric acid concentration compared to the paper support in the absence of the mixed (unsubstituted) cucurbiturils and polyvinyl alcohol coating. The effect of mixed (unsubstituted) cucurbiturils on malodour reduction was determined both with and without polyvinyl alcohol. In order to compare the different coatings, the malodour reduction R was divided by the amount of mixed (unsubstituted) cucurbiturils on each substrate sample.

TABLE 4
Malodour reduction of mixed (unsubstituted) cucurbiturils on a paper substrate measured
by GC-HS.

| | Cucurbituril | Peak | Standard | R vs. | (R vs. Support)/Cucurbituril |
|---|---|---|---|---|---|
| | (g) | area | deviation | Support (%) | (%/g) |
| Paper (Support) | 0 | 9492 | 835 | | |
| Cucurbituril: water paper | 0.166 | 1653 | 71 | 83 | 498 |
| Cucurbituril: polyvinyl alcohol | 0.211 | 2038 | 792 | 79 | 372 |
| (low MW) | | | | | |
| Cucurbituril: polyvinyl alcohol | 0.319 | 444 | 2067 | 95 | 299 |
| (high MW) | | | | | |

The presence of mixed (unsubstituted) cucurbiturils, either in coated form with polyvinyl alcohol or as a powder significantly reduces the malodour concentration in the headspace. The efficiency of malodour reduction per gram of mixed (unsubstituted) cucurbiturils in the presence and absence of binder is given in order to provide further comparison.

ii) Sponge Substrate

The results are presented in Table 5 and expressed in terms of the percentage reduction (R) in n-butyric acid concentration compared to the sponge support in the absence of mixed (unsubstituted) cucurbiturils and polyvinyl alcohol coating. The effect of mixed (unsubstituted) cucurbiturils on malodour reduction was determined both with and without polyvinyl alcohol. In order to compare the different samples, the malodour reduction R was divided by amount of mixed (unsubstituted) cucurbiturils on each sample.

TABLE 5
(Mal)odour reduction of mixed (unsubstituted) cucurbiturils on sponge substrate measured
by GC-HS

| | Cucurbituril | Peak | Standard | R vs Support | (R vs Support)/Cucurbituril |
|---|---|---|---|---|---|
| | (g) | area | Dev | (%) | (%/g) |
| Control sponge | 0 | 7724 | 817 | | |
| Cucurbituril: water sponge | 0.092 | 1540 | 320 | 80 | 870 |
| Cucurbituril: polyvinyl alcohol | 0.111 | 547 | 255 | 93 | 837 |
| (low MW) sponge | | | | | |
| Cucurbituril: polyvinyl alcohol | 0.158 | 1096 | 33 | 86 | 543 |
| (low MW) sponge | | | | | |

The presence of mixed (unsubstituted) cucurbiturils, either in coated form (with polyvinyl alcohol) or as a suspension in water (without polyvinyl alcohol) reduces the malodour concentration in the headspace by approximately 86%. The efficiency of malodour reduction per gram of mixed (unsubstituted) cucurbiturils is similar in the presence and absence of polyvinyl alcohol.

iii) No substrate

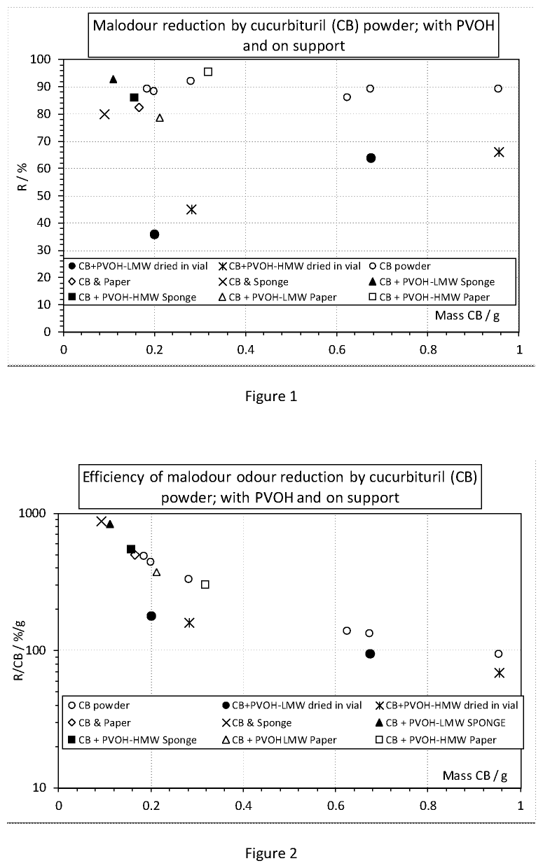

Malodour reduction experiments were performed in the absence of a substrate expanding the levels of mixed (unsubstituted) cucurbiturils used up to 1 g, with malodour reduction results summarised in Table 6. The data in Table 6 is compared with that in Tables 4 and 5 graphically in FIG. 1 which shows R (%) versus mass of mixed (unsubstituted) cucurbiturils (g) and in FIG. 2 which shows R/mass of mixed (unsubstituted) cucurbiturils (%/g) versus mixed (unsubstituted) cucurbiturils (g).

With reference to FIG. 1, the mixed (unsubstituted) cucurbiturils and polyvinyl alcohol without a substrate (filled symbols) are much less efficient in malodour reduction than free mixed (unsubstituted) cucurbiturils or mixed (unsubstituted) cucurbiturils and polyvinyl alcohol coated onto the cardboard or sponge substrates. Polyvinyl alcohol appears to have no effect on malodour reduction when the mixed (unsubstituted) cucurbiturils and polyvinyl alcohol has been coated onto a substrate.

With reference to FIG. 2, malodour reduction of mixed (unsubstituted) cucurbiturils immobilised on the cardboard or sponge substrates is indistinguishable from that of free mixed (unsubstituted) cucurbiturils (without substrate).

TABLE 6
GC-HS (mal)odour analysis of controls (no substrates).

| | Cucurbituril | Peak | Standard | R vs control | R/Cucurbituril |
|---|---|---|---|---|---|
| | (g) | area | Dev | (%) | (%/g) |
| N-Butyric acid | 0 | 31332 | 2660 | | |
| Polyvinyl alcohol low MW dried in vial | 0 | 30918 | 3412 | 1 | |
| Polyvinyl alcohol high MW dried in vial | 0 | 29838 | 503 | 5 | |
| Cucurbituril + polyvinyl alcohol low MW | | | | | |
| dried in vial | 0.200 | 20208 | 1731 | 36 | 178 |
| Cucurbituril + polyvinyl alcohol high | 0.282 | 17312 | 946 | 45 | 159 |
| MW dried in vial | | | | | |
| Cucurbituril + polyvinyl alcohol low MW | 0.675 | 11327 | 275 | 64 | 95 |
| dried in vial | | | | | |
| Cucurbituril + polyvinyl alcohol high | 0.955 | 10720 | 1229 | 66 | 69 |
| MW dried in vial | | | | | |
| Cucurbituril powder | 0.185 | 3328 | 2007 | 89 | 483 |
| Cucurbituril powder | 0.200 | 3799 | 1438 | 88 | 439 |
| Cucurbituril powder | 0.282 | 2470 | 1431 | 92 | 327 |
| Cucurbituril powder | 0.625 | 4292 | 706 | 86 | 138 |
| Cucurbituril powder | 0.675 | 3591 | 1134 | 89 | 131 |
| Cucurbituril powder | 0.955 | 3456 | 1159 | 89 | 93 |

### Example 2 Demonstrates that Mixed (Unsubstituted) Cucurbiturils May be Constrained on a Cardboard or Sponge Substrate without Detriment to Malodour Performance Efficiency

### Example 3: Cucurbituril Agglomerates

(a) Sample Preparation

Mixed (unsubstituted) cucurbituril agglomerates were produced on a fluidized bed spray granulator laboratory unit type Glatt, with inlet temperature 100-130° C., and inlet air flow 60−130 m³/h. polyvinyl pyrrolidone (PVP Luvitex K30) and polyvinyl alcohol (PVOH Poval 4−88) were used as binders. The mixed (unsubstituted) cucurbituril powder was introduced into the preheated chamber and water or aqueous binder solution was sprayed onto the powder to form mixed (unsubstituted) cucurbituril agglomerates of 1.0-3.15 mm diameter. Details on the cucurbituril agglomerates are summarised in Table 7.

TABLE 7
Mixed (unsubstituted) cucurbituril agglomerates produced by fluidized bed drying. Sample
3 was produced by adding water to powder mixed in an orbital mixer. Sample 7 was sieved to
separate small particles with diameter <1 mm (Sample 7b). PVP = polyvinyl pyrrolidone; PVOH =
polyvinyl alcohol.

| | | | Composition | |
|---|---|---|---|---|
| Name | Size (mm) | Binder | Cucurbituril | Binder |
| Sample 1 | 1.0-3.15 | PVP | 96% | 4% |
| Sample 2 | | PVP | 94% | 6% |
| Sample 3 | | Without | 100% | — |
| Sample 4 | | Without | 100% | — |
| Sample 5 | | PVP | 90% | 10% |
| Sample 6 | | PVOH | 95% | 5% |
| Sample 7a | | PVOH | 90% | 10% |
| Sample 7b | 0.63-1.0 | PVOH | 90% | 10% |

Mixed (unsubstituted) cucurbituril-coated glass beads were produced on a fluidized bed spray granulator laboratory unit type Glatt, with inlet temperature 100−130° C., and inlet air flow 80−120 m³/h. The glass beads (Poraver, with diameter 0.5−1 mm, or 1.0−2.0 mm), 1:1 mass ratio with mixed (unsubstituted) cucurbituril, were introduced into the preheated chamber, and an aqueous suspension of mixed (unsubstituted) cucurbituril and binder (45% w/w solids) was sprayed onto the beads. Details on the cucurbituril-coated glass beads are summarised in Table 8.

TABLE 8
Overview of mixed (unsubstituted) cucurbituril-coated glass beads produced by fluidised
bed drying.

| | | | Agglomerates (% w/w) | | |
|---|---|---|---|---|---|
| Name | Bead Size (mm) | Binder | Glass Beads | Cucurbituril | Binder |
| Sample 8 | 0.5-1 | PVP | 49.25 | 49.25 | 1.5 |
| Sample 9 | | PVP | 48.5 | 48.5 | 3 |
| Sample | 1.0-2.0 | PVP | 48.5 | 48.5 | 3 |

(b) (Mal)Odour Reduction: Gas Chromatography-Headspace Analysis (GC-HS)

The ability of the mixed (unsubstituted) cucurbituril agglomerates and mixed (unsubstituted) cucurbituril-coated beads to absorb undesirable target compounds was determined by GC-HS.

The model malodour compounds were n-butyric acid, benzene or ethyl-benzene. 4 μL (microliter) of each malodour compound was placed in a 1.5 mL vial inside a 20 mL headspace vial containing the mixed (unsubstituted) cucurbituril agglomerates or mixed (unsubstituted) cucurbituril-coated beads. The malodour compound concentration was measured by gas chromatography-headspace analysis (GC-HS). Analysis used a 60 metre DB-wax column (Agilent Technologies) with samples equilibrated for 30 minutes in the headspace oven at 90 degrees centigrade (n-butyric acid only) or 32 degrees centigrade (n-butyric acid, benzene, and ethyl-benzene). 10 mL of headspace gas was extracted for analysis. All measurements were performed in triplicate. The results are summarised in Tables 9 to 11.

The malodour compound concentration was determined by integrating the peak area detected at the characteristic retention time for the malodour compound. The reduction was calculated as the ratio of the malodour compound peak in the presence of the mixed (unsubstituted) cucurbituril agglomerates or mixed (unsubstituted) cucurbituril-coated beads, relative to the control peak (no cucurbituril). Samples were compared using a consistent amount of mixed (unsubstituted) cucurbituril with a 1:10 mass ratio of (mal)odour compound to mixed (unsubstituted) cucurbituril.

TABLE 9
Performance of mixed (unsubstituted) cucurbituril agglomerates and mixed
(unsubstituted) cucurbituril-coated beads at reducing n-butyric
acid concentration, measured by GC-HS.

| | Sample | % reduction | |
|---|---|---|---|
| Sample | weight (mg) | 90°C. | 32° C. |
| N-butyric acid (control) | n/a | 0 ± 16 | 0 ± 16 |
| Cucurbituril powder | | 67 ± 10 | 71 ± 13 |
| Sannple 3 | 40 | 52 ± 1 | 50 ± 11 |
| Sannple 4 | | 40 ± 2 | 40 ± 9 |
| Sannple 5 | | 60 ± 6 | 72 ± 8 |
| Sannple 6 | | 56 ± 2 | 49 ± 11 |
| Sample 7a | | 46 ± 6 | 30 ± 9 |
| Sample 7b | | 46 ± 3 | 21 ± 4 |
| Poraver 0.5-1.0nnnn | | 8 ± 2 | 14 ± 3 |
| Sannple 8 | 80 | 51 ± 2 | 47 ± 6 |
| Sannple 9 | | 60 ± 13 | 53 ± 11 |
| Poraver 1.0-2.0nnnn | 40 | 12 ± 1 | 15 ± 1 |
| Sample 10 | 80 | 57 ± 4 | 50 ± 8 |

TABLE 10
Performance of mixed (unsubstituted) cucurbituril agglomerates
and mixed (unsubstituted) cucurbituril-coated beads at
reducing benzene concentration, measured by GC-HS.

| Sample | Sample weight (mg) | % reduction |
|---|---|---|
| Benzene (control) | n/a | 0 ± 9 |
| Cucurbituril powder | 40 | 17 ± 1 |
| Sample 6 | | 12 ± 1 |
| Sample 10 | 80 | 13 ± 1 |

TABLE 11
Performance of mixed (unsubstituted) cucurbituril agglomerates and mixed
(unsubstituted) cucurbituril-coated beads at reducing ethylbenzene concentration, measured by
GC-HS.

| Sample | Sample weight (mg) | % reduction |
|---|---|---|
| Ethylbenzene (control) | n/a | 0 ± 13 |
| Cucurbituril powder | 40 | 11 ± 2 |
| Sannple 2 | | 10 ± 3 |
| Sannple 9 | 80 | 25 ± 4 |

Mixed (unsubstituted) cucurbituril agglomerates and mixed (unsubstituted) cucurbituril-coated glass beads absorb malodour compounds. Absorption occurs at a range of temperatures.

1. A method for reducing malodour comprising the step of providing a solid composition comprising one or more cucurbiturils and derivatives and/or analogues thereof suspended in and/or bound by a thermoplastic and/or a thermosetting polymer medium, wherein the source of the malodour is external.

2. The method according to claim 1, wherein the cucurbituril is selected from any one of the group consisting of cucurbit[5]uril, cucurbit[6]uril, cucurbit[7]uril, cucurbit[8]uril, and a mixture thereof.

3. The method according to claim 2, wherein the concentration of cucurbit[5]uril is from about 0 to about 99% by weight, based on the total weight of cucurbituril in the composition

4. The method according to claim 2, wherein the concentration of cucurbit[6]uril is from about 0.1 to about 99% by weight, based on the total weight of cucurbituril in the composition.

5. The method according to claim 2, wherein the concentration of cucurbit[7]uril is from about 0.1 to 99% by weight, based on the total weight of cucurbituril in the composition.

6. The method according to claim 2, wherein the concentration of cucurbit[7]uril is less than 45% by weight, based on the total weight of cucurbituril in the composition.

7. The method according to claim 2, wherein the concentration of cucurbit[8]uril is from about 0.1 to 99% by weight, based on the total weight of cucurbituril in the composition.

8. The method according to claim 2, wherein the total concentration of cucurbit[5]uril, cucurbit[6]uril, cucurbit[7]uril, and cucurbit[8]uril in the composition is greater than 75% by weight, based on the total weight of cucurbituril in the composition.

9. The method according to claim 2, wherein the composition comprises 1 to 17% by weight of cucurbit[5]uril, 30 to 50% by weight of cucurbit[6]uril; 20 to 37% by weight of cucurbit[7]uril, 10 to 27% by weight of cucurbit[8]uril, and less than 1% by weight of cucurbit[4]uril, cucurbit[9]uril and/or higher molecular weight cucurbiturils, based on the total weight of cucurbituril in the composition.

10. The method according to claim 1, wherein the thermoplastic medium is selected from the group consisting of linear low density polyethylene, low density polyethylene, medium density polyethylene, high density polyethylene, polyethylenes, polypropylenes, polyesters, polyethylene terephthalate, polyacrylate homo- and co-polymers, polymethacrylate homo- and co-polymers, poly(methyl methacrylate), poly(acrylonitrile butadiene styrene), polyamides, poly(lactic acid), poly(benzimidazole), polycarbonates, poly(ether sulfone), poly(oxymethylene), poly(etherether ketone), poly(etherimide), polystyrene, polyvinyl chloride, polyvinylidene fluoride, polytetrafluroethylene, celluloses, polysaccharides, polyvinyl alcohol, polyvinyl acetate, partially hydrolysed polyvinyl acetate, polyvinyl pyrrolidone, and mixtures thereof.

11. The method according to claim 1, wherein the thermosetting medium is selected from the group consisting of polyurethanes, polyurea polyurethane hybrids, vulcanized rubber, polyacrylates, polymethacrylate, phenol-formaldehyde resins, urea-formaldehyde resins, melamine-formaldehyde resins, epoxy resins, benzoxazines and hybrids thereof with epoxy and phenolic resins, polyimides, polybismaleimides, cyanate ester resins, furan resins, silicone resins, vinyl ester resins, alkyd resins, and mixtures thereof.

12. The method according to claim 1, wherein the composition additionally comprises one or more fragrance molecules.

13. The method according to claim 1, wherein the solid composition is in the form of an isolated film, a coating adhered to an inanimate surface, a porous substrate or an agglomerate.

14. The method according to claim 13, wherein
when the solid composition is in the form of an isolated film, the composition comprises 0.01 to 10% w/w one or more cucurbiturils and derivatives and/or analogues thereof.

15. The method according to claim 13, wherein when the solid composition is in the form of an isolated film, the one or more cucurbiturils and derivatives and/or analogues thereof are in the form of particles or agglomerates of particles of D90 no larger than the film thickness.

16. The method according to claim 13, wherein when the solid composition is in the form of a coating, the composition comprises more than 10% w/w one or more cucurbiturils and derivatives and/or analogues thereof and optionally one of more of carbon black and/or pigment and/or pigments extender and derivatives and/or analogues thereof, wherein the solid composition comprises at least an effective amount of cucurbiturils and derivatives and/or analogues thereof.

17. A solid composition comprising one or more cucurbiturils and derivatives and/or analogues thereof suspended in a thermoplastic and/or a thermosetting polymer medium, the solid composition further comprising one or more fragrance molecules.

18. The solid composition according to claim 17 in the form of an isolated film, a coating adhered to an inanimate surface, or a porous substrate.

19. The solid composition according to claim 17, wherein when the solid composition is in the form of an isolated film, the one or more cucurbiturils and derivatives and/or analogues thereof are in the form of particles of D90 no larger than the film thickness.

20. A solid composition comprising one or more cucurbiturils and derivatives and/or analogues thereof bound by a thermoplastic and/or a thermosetting polymer medium, wherein when the solid composition is in the form of a coating adhered to an inanimate surface or an agglomerate, the composition comprises more than 10% w/w one or more cucurbiturils and derivatives and/or analogues thereof and optionally one or more of carbon black and/or inorganic pigment and/or pigment extender and derivatives and/or analogues thereof, wherein the solid composition comprises at least an effective amount of the one or more cucurbiturils and derivatives and/or analogues thereof.
